(19) United States
(12) Patent Application Publication
(10) Pub. No.: US 2012/0239140 A1
(43) Pub. Date: Sep. 20, 2012

(54) MEDICAL PRODUCT COMPRISING AN ACTIVE COATING

(76) Inventors: Eric Wittchow, Nuernberg (DE); Klaus Groschner, Hart bei Graz (AT); Marlen Braune, Marloffstein (DE)

(21) Appl. No.: 13/421,343

(22) Filed: Mar. 15, 2012

Related U.S. Application Data
(60) Provisional application No. 61/453,146, filed on Mar. 16, 2011.

(51) Int. Cl. A61F 2/82; B05D 7/00

(52) U.S. Cl. 623/1.46; 427/2.1

(57) ABSTRACT

An embodiment of the present invention relates to a medical product coated with a polymer layer, which comprises an inhibitor of the TRPC-3 ion channel underneath, in, and/or on the biostable or biodegradable polymer layer.

### CROSS REFERENCE

The present application claims priority on co-pending U.S. Provisional Application No. 61/453,146 filed on Mar. 16, 2011; which application is incorporated by reference.

### TECHNICAL FIELD

The present invention relates to a medical product with an active coating. One example embodiment is coated with a polymer layer and comprises underneath, in, and/or on the biostable or biodegradable polymer layer a selective inhibitor of the TRPC-3 ion channel within the TRP family.

### BACKGROUND

A wide variety of medical endoprostheses or medical products or implants for highly diverse applications are known from the prior art. They are used e.g. to support vessels, hollow organs, and ductal systems (endovascular implants), to attach and temporarily affix tissue implants and tissue transplants, and for orthopedic purposes such as pins, plates, or screws.

Stents are used in a therapy that is frequently applied at this time, particularly in the case of cardiovascular diseases. They are used to hold hollow organs open. They often comprise a body in the form of a possibly filigree, tubular or hollow cylindrical matrix lattice which is open at both longitudinal ends. The tubular matrix lattice of an endoprosthesis of this type is inserted into the hollow organ to be treated, where it supports the hollow organ to be treated. Stents have become established for use to treat vascular diseases in particular. Insertion of stents, possibly with the aid of a balloon catheter, for example, allows constricted regions in blood vessels to be expanded and held open, thereby increasing the lumen. Stents can also be used in cancer treatment, for example, to hold open constrictions caused by malignant tumors in respiratory passages (e.g. the trachea), bile ducts, or the esophagus after expansion thereof.

Although the use of stents or other medical products makes it possible to obtain an optimal cross section of the hollow organ that is necessary primarily for therapeutic success, the permanent presence of such a foreign body can initiate a cascade of biological processes that promote e.g. inflammation of the hollow organ to be treated, or a necrotic change, and that can cause a gradual closure of the stented blood vessel. In the worst case, this change in the hollow organ can result in restenosis or even closure of the hollow organ.

It is desirable to avoid the above-described, inflammation-promoting effect of medical products to the greatest extent possible in the future, since the result is diminished efficacy of the medical product and the possibility of further damage to the organism being treated.

Recent attempts have been made to reduce the risk of restenosis associated with the implantation of stents by applying special coatings. The coating systems themselves are used in part as a supporting matrix in which one or more medicinal agents are embedded (local drug delivery). The coating typically covers at least one surface of the endovascular implant facing the vessel wall.

The coatings are composed of a biocompatible material which is either of natural origin or can be obtained synthetically. Biodegradable coating materials ensure particularly good compatibility and provide a way to influence the elution characteristics of the embedded medicinal agent.

Numerous studies have since confirmed the positive effect of biocompatible coatings on restenosis tendency in the case of metallic stents. Despite these successes, a considerable risk of restenosis also remains with the materials used so far.

### SUMMARY

One of the problems addressed by the invention is that of providing a medical product that reduces or prevents increased cellular proliferation of smooth muscle cells by the use of a high-affinity and selective inhibitor of the TRPC-3 ion channel. . These are just some of the important benefits and advantages achieved through invention embodiments.

This and other problems are solved by embodiments of the invention that provide a medical product which is coated, entirely or in parts, with a biostable or biodegradable polymer layer, and which comprises an inhibitor of the TRPC-3 ion channel underneath, in, and/or on the biostable or biodegradable polymer layer.

### BRIEF DESCRIPTION OF THE DRAWINGS

FIG. 1 shows a diagram illustrating the activation pattern of the TRPC ion channels after mechanical tissue overexpansion which normally occurs with stent implantation using a commercially available cobalt chromium stent.

FIG. 2 shows a diagram that illustrates the influence of a stent according to the invention comprising a TRPC-selective inhibitor (Pyr3) compared to an uncoated control stent (316L) on the important proliferation marker (hKI-67) and the differentiation of smooth muscle cells (hSMTN) and the total number of muscle cells (ALPHA-actin) containing α-actin.

FIG. 3 shows a diagram which illustrates the influence of a stent according to the invention comprising a TRP-selective inhibitor (Pyr3) compared to an uncoated control stent (316L) on the one further proliferation marker (PCNA).

### DETAILED DESCRIPTION

Highly promising approaches to reducing the risk of restenosis involve coated, drug-eluting stents which, due to the active agent, influence the most important cellular functions.

This is accomplished, inter alia, by activating or inhibiting ion channels which control the various cellular functions and are present in the cell membrane. One of the most extensive families of cellular ion channels are the TRP ion channels (Transient Receptor Potential) with the subfamily TRPC ion channels (Transient Receptor Potential Classical or Canonical).

Almost all members of the TRPC family are also expressed in the smooth muscle cells of the vascular musculature. The smooth muscle cells are a decisive factor in a restenosis. Due to tissue irritation that occurs during implantation, a repair mechanism is triggered in these cells that results in increased proliferation of the cells. It has been discovered, however, that the complex process of restenosis can be advantageously influenced by way of a specific blockade especially of ion channels associated with proliferation.

By the local application of selective inhibitors having a high affinity to the cellular TRPC-3 ion channels, it was demonstrated for the first time that the proliferation of smooth muscle cells after stent implantation could be clearly reduced. This result is even more surprising since other calcium channel blockers that do not act selectively on one of the TRPC channels, but rather follow another mechanism of action, do not have an antirestenotic effect in clinical application.

In the following, the term “inhibitor” is used as the equivalent of “inhibitor of the TRPC-3 ion channel”.

An inhibitor within the scope of the present invention is an inhibitory substance that influences one or more reactions—of a chemical, biological, or physiological nature—such that they are slowed, inhibited, or prevented. For example, one feature of a TRPC-3 inhibition is the prevention of calcium entry into the cell. One example inhibitor is an antiproliferative active agent, i.e., an active agent that inhibits vascular growth by suppressing expansion-induced NF-AT activation.

In an example embodiment of the present invention, the inhibitor is an amido-phenyl-pyrazole or a derivative thereof, in particular, an amido-phenyl-5-trifluoromethylpyrazole or a derivative thereof. Particularly preferably, the inhibitor is ethyl-14442,3,3-trichloroacrylamie)phenyl)-5-(trifluoromethyl)-1H-pyrazole-4-carboxylate (Pyr3).

Derivatives of amido-phenyl-5-trifluoromethylpyrazole include, but are not limited to, an amido-phenyl-pyrazole which additionally carries one or more different or identical substituents on the pyrazole ring and/or the amide function. The substituents include, but are not limited to, trifluoromethyl-, phenyl-, and monohalogenated or polyhalogenated derivates thereof, trichloroethylene, pyridinyl and halogenate derivatives thereof, and 5- or 6-membered heterocycles which can contain one or more heteroatoms such as N, S or O. Advantageously, the substituent(s) is/are selected such that the lipophilicity of the amidopyrazole is increased, to better control the elution of the active agent(s) out of the polymeric supporting matrix, and to increase the tissue retention time.

Surprisingly, it has been shown that the proliferation of smooth muscle cells can be inhibited by applying a selective inhibitor of the TRPC-3 ion channel, in particular Pyr3, underneath, in, and/or on the bio stable or biodegradable polymer layer. Due to the high affinity of Pyr3 to the TRPC-3 ion channel, it is therefore possible to provide a medical product that has a lower risk of restenosis after implantation, since the proliferation of smooth muscle cells is effectively inhibited.

Medical products within the scope of protection of the present inventions are any types of medical devices used, at least in part, for implantation in the body of a patient. Examples include, but are not limited to, implantable devices, cardiac pacemakers, catheters, needle injection catheters, blood clot filters, vascular transplants, balloons, stent transplants, biliary stents, intestinal stents, bronchial stents, esophageal stents, ureteral stents, aneurysm-filling coils and other coil devices, transmyocardial revascularization devices, percutaneous myocardial revascularization devices. Furthermore, any natural and/or artificial medical products can be used, such as prostheses, organs, vessels, aortas, heart valves, tubes, organ replacement parts, implants, fibers, hollow fibers, membranes, banked blood, blood containers, titer plates, adsorber media, dialyzers, connecting pieces, sensors, valves, endoscopes, filters, pump chambers, and other medical devices intended to have hemocompatible properties. The expression “medical products” is broad and refers in particular to products that come in contact with blood briefly (e.g., endoscopes) or permanently (e.g., stents).

Particularly preferred medical products are balloon catheters and endovascular prostheses, in particular stents.

Stents of a conventional design have a filigree support structure composed of metallic struts. The support structure is initially provided in an unexpanded state for insertion into the body, and is then widened into an expanded state at the application site. The stent can be coated before or after it is crimped onto a balloon.

The base body of the stent comprises or consists of one or more metals such as iron, magnesium, nickel, tungsten, titanium, zirconium, niobium, tantalum, zinc, platinum, iridium; or silicone. There can optionally be a second component comprising one or more metals such as lithium, sodium, potassium, calcium, manganese, iron, or tungsten. One desirable material is a zinc-calcium alloy.

In a further embodiment, the base body is made of a memory effect material one or more o nickel-titanium alloys, copper-zinc-aluminum alloys, or combinations thereof. One preferred material is Nitinol®.

In a further preferred embodiment, the base body of the stent comprises stainless steel, preferably a Cr—Ni—Fe steel, more preferably 316L alloy, or a Co—Cr steel. Furthermore, the base body of the stent comprises at least in part, of plastic and/or a ceramic.

Additionally, a passivating silicon carbide layer (SiC) can be provided on the base body of the stent comprising a metallic material. It can be applied using a method known to persons skilled in the art, and it is located underneath the layer containing the inhibitor of the TRPC-3.

In a further embodiment, the base body of the stent is composed of a biocorrodible metallic material, such as a biocorrodible alloy including, but not limited to, magnesium, iron, and tungsten. In one embodiment, the biocorrodible metallic material is a magnesium alloy.

A biocorrodible magnesium alloy is understood to be a metallic microstructure having magnesium as the main component. The main component is the alloy component that comprises the largest percentage by weight of the alloy. A percentage of the main component is preferably more than about 50% by weight, in particular more than about 70% by weight. Preferably, the biocorrodible magnesium alloy contains yttrium and other rare-earth metals, since an alloy of that type is characterized by its physical-chemical properties and high biocompatibility, in particular also the breakdown products thereof. Particularly preferably, a magnesium alloy is used which comprises about 5.2 to about 9.9% by weight of rare-earth metals, about 3.7 to about 5.5% by weight yttrium, and <about 1% by weight other materials. Magnesium makes up the remainder of the alloy to reach 100% by weight. This magnesium alloy has been shown to exhibit high biocompatibility, favorable processing properties, good mechanical characteristic values, and a corrosion behavior that is adequate for the intended uses. The term “rare-earth metals” refers to scandium (21), yttrium (39), lanthanum (57) and the 14 elements following lanthanum (57), namely cerium (58), praseodymium (59), neodymium (60), promethium (61), samarium (62), europium (63), gadolinium (64), terbium (65), dysprosium (66), holmium (67), erbium (68), thulium (69), ytterbium (70), and lutetium (71).

In a further embodiment, the stent comprises natural polymers such as collagen, chitin, chitosan, heparin. In a further embodiment, the stent is made of degradable polymers, e.g., a polylactic acid such as PDLA, PLLA, PLGA, and P3HB, P4HB, and copolymers.

The stent design is preferably adapted such that contact with the vessel wall is maximized. This promotes uniform elution of the pharmacological active agent.

At least a portion of the surface of the medical product (e.g., the balloon or stent), and/or the base body thereof (made of a metallic material of one or more metals, or of a biocorrodible metallic material, or a polymer), comprises a biostable polymer or a biodegradable polymer layer which contains an inhibitor of the TRPC-3 ion channel. It is preferably an amido-phenyl-pyrazole or a derivative thereof, more preferably an amido-phenyl-5-trifluoromethylpyrazole or a derivative thereof, most preferably Pyr3.

The expression “coating” or “polymeric supporting matrix” is used within the scope of the present invention as a synonym for the biostable or biodegradable polymer layer.

A biostable polymer layer or a biodegradable polymer layer within the scope of the invention is an application, at least in sections, of the components of the coating onto the medical product. Preferably, the coating covers the entire surface of the medical product. The layer thickness is preferably in the range of about 0.5 μm to about 30 μm, and more preferably about 1 μm to about 12 μm.

The weight component of a polymeric supporting matrix according to the invention relative to the components of the coating forming the coating is preferably at least about 40%, particularly preferably at least about 70%. The weight component of the inhibitor relative to the components of the coating forming the coating preferably does not exceed about 30%, and more preferably does not exceed about 15%.

The coating can be applied directly to the medical product. The processing can be performed using standard methods for the coating. Single-layered systems or multiple-layered systems (e.g., base coat layers, drug coat layers, or top coat layers) can be created. The coating can be applied directly to the base body of the implant, or further layers can be provided therebetween to promote adhesion, for example.

As an alternative, the biostable or biodegradable polymer layer containing the inhibitor can be present as a cavity filling or a component of a cavity filling. The medical product, in particular the stent, comprises one or more cavities for this purpose. Cavities are located on the surface of the medical product, for example, and can be created using laser ablation, for instance, in in a size ranging from nanometers to micrometers. In the case of medical products, in particular stents, comprising a biodegradable base body, a cavity can also be disposed in the interior of the base body. In that case, the material is not released until exposure thereof, which in some (but not all) embodiments will not occur until the base body is degraded to expose the interior cavity. Put another way, in some (but not all) embodiments the cavity is an interior cavity that is protected from exposure to the surrounding environment by the base body and is not exposed to the exterior environment until the base body corrodes to expose it. In regard to the design of the cavity, a person skilled in the art can refer to the systems described in the prior art. In that particular case, the expression “cavity” refers to holes and recesses, for example.

The polymer layer comprises resorbable, biodegradable polymers or non-resorbable, biostable polymers.

Most preferably, the biodegradable polymer layer is selected from polydioxanones, polyglycolides, poly-L-, poly-D-, and poly-D,L-lactide, and poly-c-caprolactone, ethyl vinyl acetate, polyphosphorylcholine, polyhydroxyvalerate, cholesterol, cholesterol ester, alginate, chitosan, levan, hyaluronic acid, uronides, heparin, dextrane, cellulose, fibrin, albumin, polypeptides, or combinations thereof, including, but not limited to copolymers, blends, and derivatives of these compounds.

Further, the polymer could also be selected from the class of biostable polymers including polypropylene, polyethylene, polyisobutylene, polybutylene, polyetheretherketone, polyethylene glycol, poly-propylene glycol, polyvinyl alcohols, polyvinyl chloride, polyvinyl fluoride, polyvinyl acetate, poly(ethyl acrylate), poly(methyl acrylate), polytetrafluorethylene, polychlorotrifluorethylene, PA 11, PA 12, PA 46, PA 66, polyamidimides, polyethersulfone, polyphenylsulfone, polycarbonates, polybutylene terephthalate, polyethylene terephthalate, elastanes, thermoplastic polyurethane elastomers such as Pellethane, silicones, polyphosphazene, polyphenylene, polymer foams (of styrolene and carbonates), polyethylene oxide, or combinations thereof, including, but not limited to copolymers, blends, and derivatives of these compounds.

The biostable or biodegradable polymer layer can be designed in accordance with the desired elution rate and the individual characteristics of the various active agents that are used, and in accordance with the different rates of resorption and degradation at the site of action of the medical product.

The inhibitor of the TRPC-3 ion channel can be applied underneath, in, and/or on the biostable or biodegradable polymer layer in a concentration between about 0.25 to about 7 μg/mm² stent surface, more preferably between about 0.6 to about 3.8 μg/mm² stent surface, or in other loading concentrations which may be, for instance, less than about 0.25 to about 7 μg/mm² stent surface or greater than about 3.8 μg/mm².

In one example embodiment, the biostable or biodegradable polymer layer is present as an abluminal coating on the stent. This has the advantage that, after implantation of the stent, the inhibitor present in the biostable or biodegradable polymer layer comes into direct contact only with the irritated tissue and can therefore apply its effect locally, and the luminal surface of the stent is free of any stimulus-eliciting components that may be present, such as polymeric supporting matrix or pharmaceutical active agent.

The medical product according to the invention can comprise other pharmaceutical active agents in addition to the inhibitor. The additional pharmaceutical active agent can be located underneath, in, and/or on the bio stable or biodegradable polymer layer. Preferably, the pharmaceutical active agent is selected from the following drug classes: antiangiogenic, antimicrobial, antimitotic, antimyotic, antineoplastic, antiphlogistic, antiproliferative, antithrombotic, and vasodilator agents, although others will prove useful and beneficial and are within the scope of the invention.

Preferably, the pharmaceutical active agent is selected from dexamethasone, methylprednisolone, diclophenac, paclitaxel, colchicine, actinomycin D, methotrexate, limus compounds, sirolimus (rapamycin), Myolimus, novolimus, zotarolimus (ABT-578), tacrolimus (FK-506), everolimus, biolimus, in particular biolimus A9, and pimecrolimus, deforolimus, novolimus, cyclosporin A, mycophenolic acid, abciximab, iloprost, simvastatin, mevastatin, atorvastatin, lovastatin, pitavastatin, pravastatin, fluvastatin, 17b-estradiol, daizein, genistein, fibrates, immunsuppressants, sartanes, calcium channel blockers, tacrolimus, imidazole, antiallergenics, decoy oligodeoxynucleotide (dODN), fibrine, steroids, proteins/peptides, analgesics, antirheumatics, bosentan, fasudil, RGD peptides and cyclic RGD (cRGD) (having the sequence Arg—Gly—Asp), organic gold or platinum compounds, triclosan, cephalosporin, aminoglycoside, nitrofurantoin, penicillins, oxacillin and sulfonamide, metronidazole, 5-fluoruracil, vinblastine, vincristine, epothilones, endostatin, verapamil, angiostatin, angiopeptin, methotrexate, colchicine, flavopiridol, suramin, clotrimazole, flucytosine, griseofulvin, ketoconazole, miconazole, nystatin, terbinafine, prednisolone, corticosterone, budesonide, estrogen, hydrocortisone, mesalamine, sulfasalazine, heparin and the derivatives thereof, urokinase, PPack, argatroban, aspirin, abciximab, synthetic antithrombin, bivalirudin, enoxoparin, hirudin, r-hirudin, protamine, prourokinase, streptokinase, warfarin, 7,3′,4′-trimethoxyflavone, dipyramidole, trapidil and nitroprussides.

The pharmaceutical active agents are used individually or in combination, in the same concentration or different concentrations.

The medical product according to the invention can comprise a further inner or outer coating(s). A further outer layer can cover the coating or cavity filling containing the inhibitor, entirely or in parts. This outer coating can contain a degrading polymer or be composed thereof, in particular polylactides, PLGA (poly(lactic-co-glycolic acid)) or

PLGA-PEG block copolymers. Optionally, a further active agent can be embedded in this additional, outer layer, which can elute freely or become released upon degradation of the outer coating.

In an example embodiment of the invention, the inhibitor is applied directly onto the implant surface, and is coated with a biostable or biodegradable polymer layer. Optionally, if the base body of the stent is composed of a metallic material, and a passivating silicon carbide layer (SiC) is present on the metallic material, the inhibitor is applied onto the SiC surface and is subsequently coated with a biostable or biodegradable polymer layer. Preferred polymers are those that degrade by hydrolysis, and the breakdown products of which contain, inter alia, one or more carboxyl functions, such as polyester and polyamides, or biostable polymers such as PTFE, Parylene, silicones, or urethanes. This polymer coating can additionally contain a further pharmaceutical active agent.

A further aspect of the invention is the use of amido-phenyl-pyrazole or a derivative thereof, in particular amido-phenyl-5-trifluoromethylpyrazole or a derivative thereof, particularly preferably ethyl-1-(4-(2,3,3-trichloroacrylamide)phenyl)-5-(trifluoromethyl)-1H-pyrazole-4-carboxylate as the inhibitor of the TRPC-3 ion channel to manufacture a medical product which is coated, entirely or in parts, with a biostable or biodegradable polymer layer, in particular balloon catheters or stents, wherein the inhibitor of the TRPC-3 channel is located underneath, in, and/or on the biostable or biodegradable polymer layer.

A further aspect of some invention embodiments is the use of amido-phenyl-pyrazole or a derivative thereof, preferably amidophenyl-5-trifluoromethylpyrazole or a derivative thereof, more preferably ethyl-1-(4-(2,3,3-trichloroacrylamide)phenyl)-5-(trifluoromethyl)-1H-pyrazole-4-carboxylate for the prophylaxis or treatment of a restenosis or an impairment of a vascular lumen in a section of a vessel.

A further aspect of some invention embodiments is the use of amido-phenyl-pyrazole or a derivative thereof, preferably amidophenyl-5-trifluoromethylpyrazole or a derivative thereof, more preferably ethyl-1-(4-(2,3,3-trichloroacrylamide)phenyl)-5-(trifluoromethyl)-1H-pyrazole-4-carboxylate as an antiproliferative active agent.

### EXAMPLE EMBODIMENT 1

A stent made of stainless steel is coated as follows:

A solution of ethyl-1-(4-(2,3,3-trichloroacrylamide)phenyl)-5-(trifluoromethyl)-1H-pyrazole-4-carboxylate (M=456.63 g/mol) in tetrahydrofuran (THF) (10% by weight) at room temperature is prepared. This solution is mixed with a second solution of polylactic acid (L210; Boehringer Ingelheim) in THF (10% by weight) at room temperature such that drug and polylactic acid have a weight-volume ratio of 1:1.

The stent is cleaned of dust and residue, and is clamped into a suitable stent coating device. Using an airbrush system (from the company EFD or Spraying System), the rotating stent is coated under constant environmental conditions (room temperature; 42% air humidity) with the gold salt/polymer mixture on one side. A stent having a length of 18 mm is coated in approximately 10 minutes when the nozzle is situated at a distance of 20 mm. Once the intended coating mass has been applied, the stent is dried for 5 minutes at room temperature; the stent is then rotated and clamped into position once more, and the uncoated side is coated in the same manner. The stent with finished coating is dried for 36 hours at 40° C. in a vacuum oven (Vakucell; the company MMM).

The layer of the applied coating is approximately 3-5 μm thick.

Ex vivo Test on the Aorta Model

The ex vivo test on pathologically altered human arteries and, in particular, on human aortas is a novel test model that makes it possible to better predict the clinical efficacy of biologically active coatings of medical implants such as stents or balloon catheters. Until now, it was not possible to perform this prediction in a reliable manner. The list of substances that have shown good efficacy in the experimental animal model but surprisingly did not reduce restenosis in clinical practice (e.g., calcium channel blockers, aspirin, heparin, pimecrolimus) is long. These failures have engendered doubt in the benefit of the standard animal models, since, despite additional serial examinations and comprehensive screenings, the clinical efficacy of a new substance is uncertain even though preclinical results were positive. Until now there was no test model available that investigated the antiproliferative effect of new drugs in the complex biological environment of an arteriosclerotic vessel. On the basis of this novel test model, it is now possible to obtain mechanistic insights into the origin of a restenosis after stent implantation and to develop a therapy specific for the cause thereof

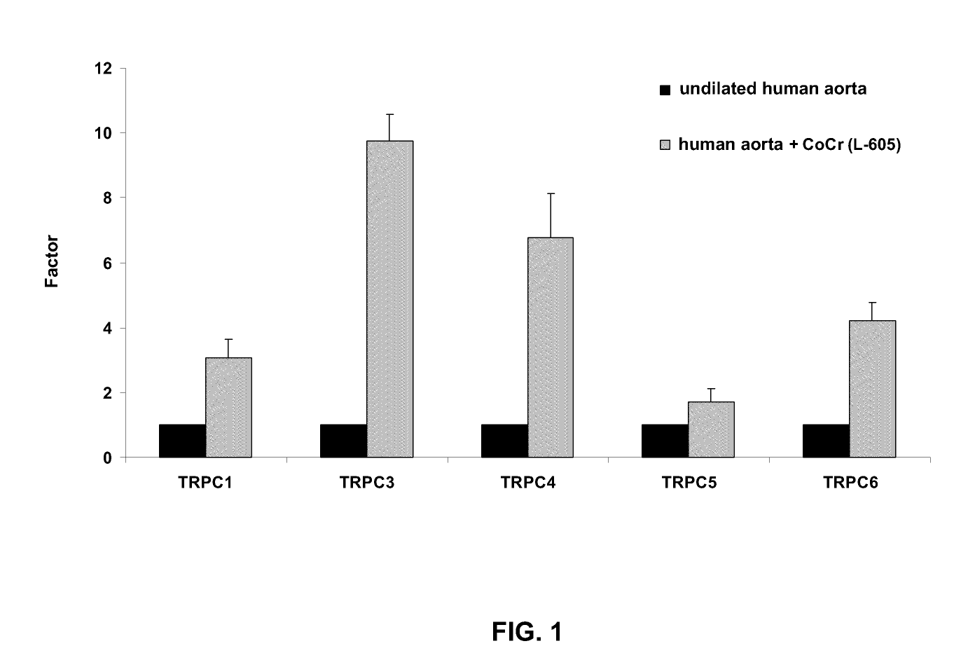

In an ex vivo experiment using pathologically altered human aortas, it was demonstrated for the first time that the activation pattern of the TRPC ion channels is changed as the result of a stent implantation and the associated tissue irritation caused by the expansion of the vessel (“stretch”) (FIG. 1). In this experiment, commercially available stents made, e.g., of a cobalt chromium alloy, were implanted in altered human aortas ex vivo, and the vessel was removed and placed in a culture bath under standard conditions for 14 days. Next, the activation of the channels TRPC-1 to TRP-C6 was determined quantitatively using PCR, and was compared with an unstented aortic region which had also been placed in the culture bath for 14 days. It is primarily the TRPC-3 ion channel, which is associated with proliferation, that is highly activated compared to an unstented control section.

As such, an ex vivo test model that overcomes the above-stated disadvantages has been developed for the first time. Ex vivo test results obtained in clinical practice were now reliably confirmed in an ex vivo test on the aortic model, in fact, much more reliably than the clinical efficacy of antiproliferative drugs and drug-eluting stent systems were able to predict previously.

The special test procedure is based on the use of human pathological vascular tissue obtained directly within one hour after operation (vasectomy). Immediately after removal, the vascular preparations are placed in a sterile phosphate buffer (PBS) at room temperature, and are transported.

In a first step, the adventitia is removed from the surrounding muscle tissue under aseptic conditions from the human, pathologically altered explants, such as aortic segments that were removed in a bypass operation, are rinsed with PCS containing standard antibiotics, and are cut lengthwise to produce strips having a length of 15-20 mm. These segments are sutured back together around a cylinder using the standard surgical technique (polyamide/nylon) such that, after the cylinder is removed, tubes having a diameter of approximately 3 mm are obtained. A 3 mm/15 stent is implanted with 14 bar into these plaque-containing, sutured explants using a balloon catheter, wherein the overexpansion of the vessel triggers a proliferative stimulus. The stented explant is now cultivated for 2 weeks in a DMEM nutritive solution without growth factors under standard conditions (increased air humidity, 5% CO2; 37° C.). Next, the stent is removed and the explant is homogenized. After a few preparation steps, a PCR is carried out.

All of the samples in FIGS. 1 to 3 were investigated using this test model.

Ex vivo Test on the Aorta Model of Embodiment 1

The human, pathologically altered explant is cut lengthwise and sutured back together around a cylinder having a diameter of 2.5 mm. Next, the stent according to the invention and embodiment 1 is inserted into the re-sutured explant, and is implanted with overexpansion. The test is then carried out as described above.

The uncoated stent made of 316L, which is unlike that according to the invention, is used as the reference.

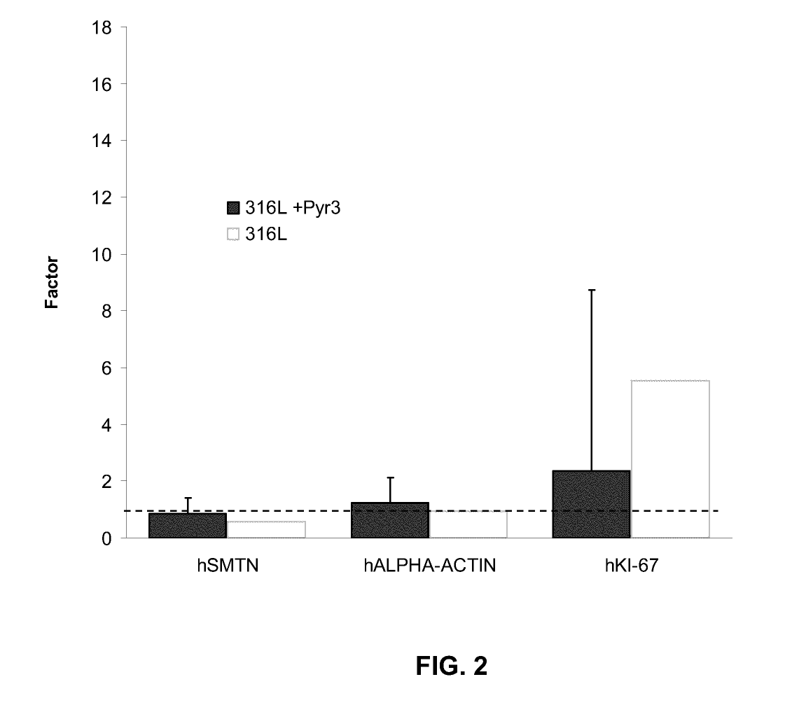

FIG. 2 shows the influence of the stent according to the invention and embodiment 1 on the proliferation and the differentiation of smooth muscle cells. The dashed line in FIG. 1 is the standard value which is 1 for an unexpanded vessel.

Tests were carried out using 3 different cell cultures: hSMTN, hALPHA-actin and hKI-67. They are:
hSMTN: Smoothelin; marker for muscle cells of the contractile phenotype
hALPHA-actin: Marker for all types of smooth muscle cells
hKI-67: Proliferation marker for monoclonal antibody KI 67

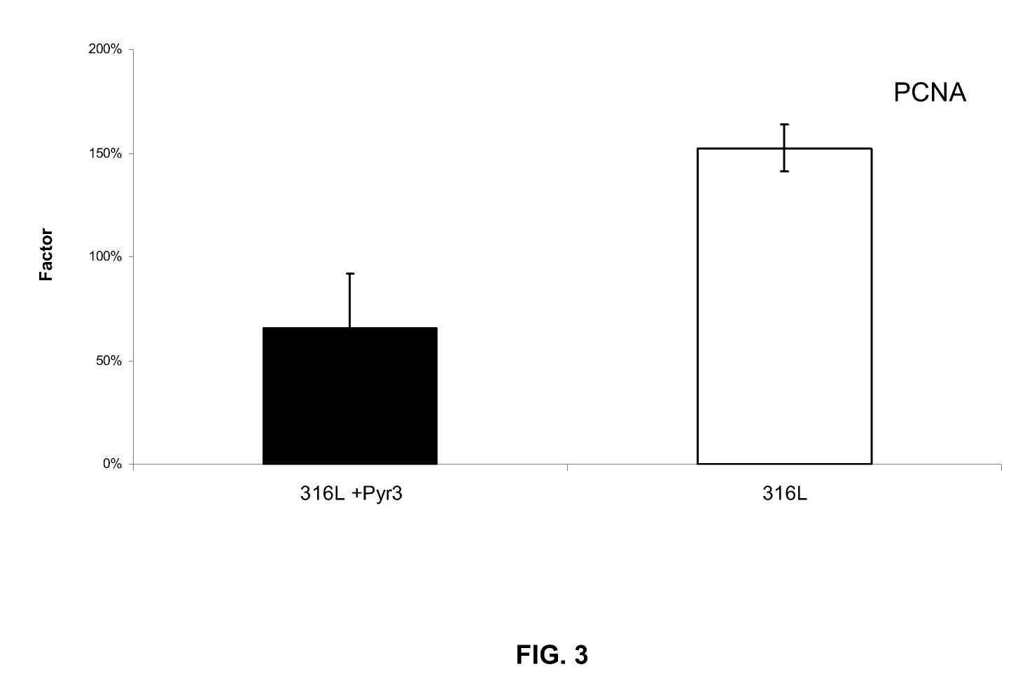

FIG. 3 illustrates the proliferation activity of the stent according to the invention on the proliferation marker PCNA.

FIG. 1 shows clearly how the mechanical expansion of the pathologically altered aorta changes the activation pattern of the various TRPC channels. The activation is determined by the extent of vascular injury, which is comparable in terms of type to a normal vascular dilation that occurs when a stenosis is treated. Based on findings so far, it is primarily the mechanosensitive channels TRPC-1 and TRPC-3 that are associated with proliferation, wherein TRPC-3 reacts most strongly to the mechanical stimulus, and the corresponding ion channel is upregulated by nearly a factor of 10 compared to an unexpanded vascular section.

FIGS. 2 and 3 show how the TRPC-3-selective inhibitor influences proliferation (measured via the two important proliferation markers KI-67 and PCNA) when it is eluted from a polymeric stent coating. A conventional stent without drug (316L) is compared in each case with the same stent comprising a selective TRPC-3 inhibitor (Pyr3) according to embodiment 1. On the basis of the expression of hSMTN and hAlpha-actin, it is clearly shown in FIG. 2 that an inhibition of the TRPC-3 ion channel with Pyr3 does not lead to the otherwise common drop in total number of smooth muscle cells after stent implantation, which is apparently why the biological stimulus to replace same is also lower, which is expressed as lower overall proliferation.

By the local application of selective inhibitors having a high affinity to the cellular TRPC ion channels, it was demonstrated for the first time that stretch-induced proliferation in a pathologically altered, human aorta after stent implantation could be clearly reduced. This result is surprising since other calcium channel blockers, which do not act selectively on one of the TRPC channels, do not have an antirestenotic effect in clinical application.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this invention.

What is claimed is:

1. A medical product, comprising:
a surface at least partially coated with a polymer layer that is biostable or biodegradable; and
comprises an inhibitor of the TRPC-3 ion channel arranged in one or more of underneath, within and over the polymer layer.

2. The medical product according to claim 1, wherein the inhibitor of the TRPC-3 ion channel is an amido-phenyl-pyrazole or a derivative thereof

3. The medical product according to claim 2, wherein the inhibitor of the TRPC-3 ion channel is ethyl-1-(4-(2,3,3-trichloroacrylamide)phenyl)-5-(trifluoromethyl)-1H-pyrazole-4-carboxylate.

4. The medical product according to claim 1, wherein the polymer layer comprises polyolefins, polyetherketones, polyethers, polyvinyl alcohols, polyvinyl halogenides, polyvinyl esters, polyacrylates, polyhalogen olefins, polyamides, polyamidimides, polysulfones, polyesters, polyurethanes, silicones, polyphosphazenes, polyphenylene, polymer foams of styrolene, polymer foams of carbonates, polydioxanones, polyglycolides, polylactides, poly-c-caprolactone, ethyl vinyl acetate, polyethylene oxide, polyphosphorylcholine, polyhydroxybutyric acids, lipids, polysaccharides, proteins, polypeptides, or combinations thereof

5. The medical product according to claim 1, wherein the inhibitor of the TRPC-3 ion channel is present in a concentration of about 0.25 to about 7 μg/mm².

6. The medical product according to claim 1, further comprising a pharmaceutical active agent that is located in one of underneath, within, and on the polymer layer.

7. The medical product according to claim 1, wherein the medical product is a balloon catheter or a stent.

8. The medical product according to claim 7, wherein the product is a stent comprising iron, magnesium, nickel, tungsten, titanium, zirconium, niobium, tantalum, zinc, platinum, iridium, silicone, lithium, sodium, potassium, calcium, manganese, copper, aluminum, chromium, cobalt, rare earth metals, stainless steel, plastic, ceramic, or combinations thereof

9. The medical product according to claim 7, wherein the polymer layer is present as an abluminal coating on the stent.

10. The medical product according to claim 1, wherein the polymer layer is the biodegradable polymer comprising polydioxanones, polyglycolides, poly-L-lactide, poly-D-lactide, poly-D,L-lactide, poly-c-caprolactone, ethyl vinyl acetate, polyphosphorylcholine, polyhydroxyvalerate, cholesterol, cholesterol ester, alginate, collogen, chitin, chitosan, levan, hyaluronic acid, uronides, heparin, dextrane, cellulose, fibrin, albumin, polypeptides, or combinations.

11. The medical product according to claim 1, wherein the polymer layer is the biostable polymer comprising polypropylene, polyethylene, polyisobutylene, polybutylene, polyetheretherketone, polyethylene glycol, polypropylene glycol, polyvinyl alcohols, polyvinyl chloride, polyvinyl fluoride, polyvinyl acetate, poly(ethyl acrylate), poly(methyl acrylate), poly(methyl methacrylate), polytetrafluoroethylene, polychlorotrifluorethylene, polyamide 11, polyamide 12, polyamide 46, polyamide 66, polyamidimides, polyethersulfone, polyphenylsulfone, polycarbonates, polybutylene terephthalate, polyethylene terephthalate, elastanes, thermoplastic polyurethane elastomers, silicones, polyphosphazenes, polyphenylene, polymer foams of styrolene, polymer foams of carbonates, polyethylene oxide, or combinations.

12. The medical product according to claim 1 further comprising a silicon carbide layer on the surface under the polymer layer.

13. The medical product according to claim 1 wherein the polymer layer has a thickness in a range of about 0.5 μm to about 30 μm.

14. The medical product according to claim 1 wherein the polymer layer is a multilayer coating.

15. The medical product according to claim 1 wherein the polymer layer is in a cavity on the surface of the support.

16. A method of making a medical device comprising:
providing a device surface;
applying a biostable polymer layer or a biodegradable polymer layer to the surface;
providing an inhibitor of TRPC-3 ion channel arranged in one of within the polymer layer, under the polymer layer, and on the polymer layer, or combinations thereof

17. The method of claim 16 wherein the inhibitor of TRPC-3 ion channel is applied to the support before the polymer layer is applied.

18. The method of claim 16 further comprising applying a passivating layer to the support before the polymer layer is applied.

19. The method of claim 16 wherein providing the inhibitor of TRPC-3 ion channel in the polymer layer comprises mixing the inhibitor with a polymer.

20. The method of claim 16 further comprising applying providing a pharmaceutical active agent in the polymer layer, under the polymer layer, or on the polymer layer, or combinations thereof
